(19) United States
(12) Patent Application Publication
(10) Pub. No.: US 2009/0206720 A1
(43) Pub. Date: Aug. 20, 2009

(54) LOW-PRESSURE GAS DISCHARGE LAMP HAVING IMPROVED EFFICIENCY

(75) Inventors: Piet Antonis, Eindhoven (NL); Ariel De Graaf, Eindhoven (NL); Spyridon Kitsinelis, Eindhoven (NL); Peter Johannes Wilhelmus Vankan, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/300,836

(22) PCT Filed: Apr. 25, 2007
(86) PCT No.: PCT/IB2007/051518

§ 371 Date: Apr. 1, 2009

(30) Foreign Application Priority Data
May 15, 2006 (EP) 06113933.3

(51) Int. Cl. H01J 61/12; H01J 61/52; H01J 1/62

(52) U.S. Cl. 313/47; 313/637; 313/484

(57) ABSTRACT

The invention relates to a low-pressure mercury vapor discharge lamp (10) comprising a metal compound which is selected from the group formed by compounds of titanium, zirconium, hafnium and their mixtures. The effect of adding the metal compound selected from said group to the gas filling of the discharge space (14) results in an increased efficiency of the low-pressure gas discharge lamp (10), because part of the emitted light from the discharge space (14) is in the visible range of the electromagnetic spectrum. In one embodiment of the invention, the low-pressure gas discharge lamp (10) produces substantially white light without the use of a luminescent layer (16) comprising a luminescent material. In another embodiment of the invention, the luminescent layer (16) is applied to the discharge vessel (12) of the low-pressure gas discharge lamp (10). The light emitted by the luminescent material can be mixed with the light emitted from the discharge space (14) to produce the required color.

### FIELD OF THE INVENTION

The invention relates to a low-pressure gas discharge lamp.

### BACKGROUND OF THE INVENTION

Low-pressure gas discharge lamps generally comprise mercury as a primary component for the generation of ultraviolet (further also referred to as UV) light. A luminescent layer comprising a luminescent material may be present on an inner wall of a discharge vessel to convert UV light into light of increased wavelength, for example, into UV-C for medical purposes, UV-B and UV-A for tanning purposes (sun tanning lamps) or into visible radiation for general illumination purposes. Such discharge lamps are therefore also referred to as fluorescent lamps. Fluorescent lamps for general illumination purposes usually comprise a mixture of luminescent materials, which determines the color of the light emitted by the fluorescent lamps. Examples of commonly used luminescent materials are, for example, a blue-luminescent europium-activated barium magnesium aluminate, BaMgAl₁₀O₁₇:Eu²⁺ (also referred to as BAM), a green-luminescent cerium-terbium co-activated lanthanum phosphate, LaPO₄:Ce,Tb (also referred to as LAP) and a red luminescent europium-activated yttrium oxide, Y₂O₃:Eu (also referred to as YOX).

The discharge vessel of low-pressure mercury vapor discharge lamps is usually constituted by a light-transmitting envelope enclosing a discharge space in a gastight manner. The discharge vessel is generally circular and comprises both elongate and compact embodiments. Normally, the means for generating and maintaining a discharge in the discharge space are electrodes arranged near the discharge space. Alternatively, the low-pressure mercury vapor discharge lamp is a so-called electrodeless low-pressure mercury vapor discharge lamp, for example, an induction lamp where energy required for generating and/or maintaining the discharge is transferred through the discharge vessel by means of an induced alternating electromagnetic field.

A drawback of the known low-pressure mercury vapor discharge lamps is that luminescent conversion efficiency is not optimal.

### SUMMARY OF THE INVENTION

It is an object of the invention to provide a low-pressure gas discharge lamp with improved efficiency.

According to a first aspect of the invention, the object is achieved with a low-pressure gas discharge lamp comprising: a light-transmitting discharge vessel enclosing, in a gastight manner, a discharge space comprising a gas filling, the gas filling comprising a metal compound, and means for maintaining a discharge in the discharge space, the metal compound being selected from the group formed by compounds of titanium, zirconium, hafnium and their mixtures.

The effect of the measures according to the invention is that the presence of the metal compound selected from the group formed by compounds of titanium, zirconium, hafnium, and their mixtures, results in an emission of light from the discharge space, of which part is in the visible range of the electromagnetic spectrum. In the discharge space of the low-pressure gas discharge lamp according to the invention, a gas discharge takes place at a low pressure. Apart from the characteristic lines of titanium, zirconium and/or hafnium atoms, the emitted light further includes a contribution from different compounds of titanium, zirconium and/or hafnium, such as chlorides, bromides, iodides and/or, for example, oxy-iodides, which are present in the discharge space, resulting in a substantially continuous spectrum of light. Part of the continuous spectrum is in the visible range. In the known low-pressure mercury vapor discharge lamps, the main emission of light is in the ultraviolet region (wavelength of the main ultraviolet light emission in the known low-pressure mercury vapor discharge lamp is at approximately 254 nanometer). To produce visible light from these known low-pressure mercury vapor discharge lamps, luminescent materials are used. The luminescent materials convert the emitted ultraviolet light into visible light. During this conversion, energy is lost which reduces the efficiency of the known low-pressure mercury vapor discharge lamps. The low-pressure gas discharge lamp according to the invention produces visible light without the need for luminescent materials, which improves the efficiency.

A further benefit of the low-pressure gas discharge lamp according to the invention is that part of the light emitted from the discharge space is in the so-called near ultraviolet region. The near ultraviolet region comprises ultraviolet-B light (further also referred to as UV-B), having a wavelength between approximately 280 nanometer and approximately 320 nanometer, and ultraviolet-A light (further also referred to as UV-A), having a wavelength between approximately 320 nanometer and approximately 400 nanometer. UV-A light and UV-B light are generally used for medical (for example, for the treatment of psoriasis), germicidal, lacquer-curing and tanning purposes. In the known low-pressure mercury vapor discharge lamps, luminescent materials are used to convert the UV-radiation of the mercury into UV-A and UV-B. However, a drawback of the known UV-generating low-pressure mercury vapor discharge lamp is that the luminescent materials used to generate UV-A and UV-B have a limited lifetime due to a relatively strong degradation of the luminescent materials. The low-pressure gas discharge lamp according to the invention emits, in addition to visible light, light in the near ultraviolet region without the use of luminescent materials. Because no luminescent materials are used, the low-pressure gas discharge lamp according to the invention has an increased efficiency and an increased lifetime compared to the known UV-generating low-pressure mercury vapor discharge lamps. To enable the emission of light in the near ultraviolet region, the discharge vessel generally is constituted of quartz or other UV transmitting materials. Furthermore, instead of the blue emission of conventional UV-emitting lamps, the low-pressure gas discharge lamp according to the invention emits, in addition to the UV-A and UV-B light, a substantially continuous spectrum of visible light covering part of the visible range of the electromagnetic spectrum.

The inventors have realized that the known low-pressure gas discharge lamps have a relatively low efficiency due to energy loss resulting from a Stokes shift. The Stokes shift is an energy loss process due to the conversion of one photon into another photon having an increased wavelength. In the known low-pressure mercury vapor discharge lamps, generally, an ultraviolet photon from the mercury vapor discharge is converted by the luminescent material into a photon of increased wavelength—for example into UV-A, UV-B and/or into visible light. The energy difference between the ultraviolet photon and the photon in the visible range is typically lost and is known as the Stokes shift. The low-pressure gas discharge lamp according to the invention produces the substantially continuous spectrum of visible light covering part of the visible range of the electromagnetic spectrum. Visible light is produced while the need for luminescent materials no longer exists, as a result of which the energy loss due to the Stokes shift has disappeared.

The use of a metal compound in a buffer gas of a low-pressure gas discharge lamp is disclosed in U.S. Pat. No. 6,972,521, in which the low-pressure gas discharge lamp has a gas discharge vessel containing a gas filling with an indium compound and a buffer gas. The gas filling with indium halides is particularly preferred. However, a drawback of using an indium compound is that the temperature inside the discharge vessel of the known low-pressure gas discharge lamp comprising indium must be relatively high to maintain sufficient indium vapor in the discharge space. This requires special adaptations to the discharge vessel when using these known low-pressure gas discharge lamps comprising indium for general illumination purposes, as these high temperatures reduce the efficiency due to the energy required to obtain the required high temperatures.

In an embodiment of the low-pressure gas discharge lamp, the gas filling further comprises a buffer gas. The buffer gas is generally constituted of an inert gas, for example, helium, neon, argon, krypton and/or xenon.

In an embodiment of the low-pressure gas discharge lamp, the metal compound comprises metal halides. In a preferred embodiment of the low-pressure gas discharge lamp, the metal halides comprise metal tetra-halides and/or metal oxy-halides. A benefit of this embodiment is that the titanium halides, zirconium halides, hafnium halides and a mixture of these halides are relatively volatile, which results in sufficient metal vapor in the gas filling of the discharge space at relatively low temperatures. For example, a low-pressure gas discharge lamp comprising titanium tetra-bromide comprises already at room temperature sufficient titanium and titanium compound in the gas filling to ensure a sufficient light emission from the discharge space of the low-pressure gas discharge lamp.

In a preferred embodiment of the low-pressure gas discharge lamp, the density of the metal compound particles, in operation, is between 5×10¹⁸ and 5×10²⁴ particles/m³. Metal compounds comprise metal atoms as well as metal molecules, which all contribute to the emission spectrum of the low-pressure gas discharge lamp according to the invention.

In an embodiment of the low-pressure gas discharge lamp, the low-pressure gas discharge lamp comprises an outer vessel enclosing the discharge vessel. A benefit of the additional outer vessel is that it provides additional thermal insulation, which further reduces the energy loss from loss of heat. Furthermore, a luminescent layer may conveniently be applied at the inside of the outer vessel, preventing the luminescent material to react with the gas filling inside the discharge vessel.

In an embodiment of the low-pressure gas discharge lamp, the low-pressure gas discharge lamp is a substantially mercury-free low-pressure gas discharge lamp. Mercury-free means that the low-pressure gas discharge lamp contains typically less than 10 microgram of mercury per lamp. The mercury in the gas filling is regarded as environmentally harmful and should therefore be avoided as much as possible. This can be achieved by replacing the mercury in a known low-pressure mercury vapor discharge lamp by metal compounds selected from the group formed by titanium compounds, zirconium compounds, hafnium compounds, and their mixtures, to obtain a low-pressure gas discharge lamp according to the invention.

In an embodiment of the low-pressure gas discharge lamp, the low-pressure gas discharge lamp comprises elements which maintain the discharge via inductive operation, further also referred to as inductive coupler. The elements may also maintain the discharge via capacitive operation, microwave operation, or via electrodes. A benefit of such a, so called electrodeless low-pressure gas discharge lamp is that the average lifetime of the electrodeless low-pressure gas discharge lamp is considerably longer compared to conventional low-pressure gas discharge lamps which have electrical contacts through the discharge vessel to transfer power into the discharge space. Generally, the electrical contacts, also referred to as electrodes, limit the lifespan of the conventional low-pressure gas discharge lamps. The electrodes may, for example, become contaminated with residue or, for example, get damaged by the discharge and cannot transfer sufficient power into the discharge space to guarantee operation of the conventional low-pressure gas discharge lamp. Providing the low-pressure gas discharge lamp with an inductive coupler according to the invention considerably increases the lifetime of the low-pressure gas discharge lamp. An example of such an inductive coupler is a coil which is, for example, arranged around the light-transmitting discharge vessel, or which is, for example, arranged in a glass protrusion, protruding into the discharge vessel. The inductive coupler may also be used, apart from to maintain the discharge, to generate the discharge in the discharge vessel of the low-pressure gas discharge lamp according to the invention.

In a preferred embodiment of the low-pressure gas discharge lamp, the discharge vessel comprises a luminescent layer comprising a luminescent material. The luminescent material, for example, absorbs part of the ultraviolet light emitted by the compounds of titanium, zirconium, hafnium, and/or their mixtures, and converts the absorbed ultraviolet light into visible light. When the low-pressure gas discharge lamp according to the invention is used for general illumination purposes, the low-pressure gas discharge lamp should produce substantially white light at the required color temperature. Due to the added titanium compounds, zirconium compounds and/or hafnium compounds, part of the light emitted by the low-pressure gas discharge lamp is in the visible range of the electromagnetic spectrum. Altering the gas-pressure and/or operating temperature inside the discharge vessel induces a change of the spectrum of the emitted light and hence also of the color of the light emitted by the low-pressure gas discharge lamp according to the invention. However, the required color temperature of the low-pressure gas discharge lamp may not be achieved by only altering the gas-pressure and/or operating temperature. Adding a luminescent layer comprising luminescent materials enables the light emitted by the luminescent material to be mixed with the light emitted from the discharge space to produce the required color temperature. Examples of commonly used luminescent materials are, for example, a blue-luminescent europium-activated barium magnesium aluminate, BaMgAl₁₀O₁₇:Eu²⁺ (also referred to as BAM) and a red-luminescent europium-activated yttrium oxysulfide, Y₂O₂S:Eu (also referred to as YOS). Although luminescent materials are used in this preferred embodiment to adapt the color of the emitted light from the low-pressure gas discharge lamp, the efficiency of the low-pressure gas discharge lamp is still higher compared to conventional low-pressure mercury vapor discharge lamps. The ultraviolet part of the light emitted by the low-pressure gas discharge lamp according to the invention is in the near ultraviolet range, which comprises a substantially longer average wavelength compared to the main ultraviolet emission of the mercury vapor (which is around 254 nanometer). This shift of the average wavelength of the ultraviolet part of the emitted light in the low-pressure gas discharge lamps according to the invention to longer wavelengths results in a reduced Stokes shift, because the difference between the average energy of the ultraviolet photon which is absorbed by the luminescent material and the average energy of the emitted photon in the visible range is reduced, resulting in a reduction of the losses. Part of the light emitted from the discharge space of the low-pressure gas discharge lamp according to the invention is in the visible range of the electromagnetic spectrum, which already improves the efficiency of the low-pressure gas discharge lamp according to the invention with respect to the conventional low-pressure mercury vapor discharge lamps. The luminescent layer comprising the luminescent material may be applied to the inside or to the outside of the discharge vessel. Applying the layer comprising a luminescent material to the outside of the discharge vessel prevents the luminescent material from reacting with the gas filling inside the discharge vessel.

In an embodiment of the low-pressure gas discharge lamp, the discharge vessel comprises a coating for thermal insulation. Generally, the operating temperature of the low-pressure discharge lamps according to the invention is higher compared to conventional low-pressure mercury vapor discharge lamps to ensure that enough metal vapor is in the gas filling. The additional coating for thermal insulation may be an infrared radiation-reflecting coating reflecting the emitted infrared radiation from the discharge space back into the discharge space. The result of the added infrared radiation-reflecting coating is an increase in temperature inside the discharge vessel. Alternatively, the coating for thermal insulation may form a shield between the increased temperature inside the discharge vessel and the outside of the discharge vessel and as such shield a user handling the low-pressure gas discharge lamps from the increased temperature.

The invention also relates to the use of the low-pressure gas discharge lamp according to the invention for diagnostics or therapeutic applications, and for germicidal or cosmetic applications. The diagnostics applications, for example, comprise medical imaging and the therapeutic applications, for example, comprise radiotherapy for the treatment of psoriasis. The cosmetic applications, for example, comprise tanning.

### BRIEF DESCRIPTION OF THE DRAWINGS

These and other aspects of the invention are apparent from and will be elucidated with reference to the embodiments described hereinafter.

In the drawings:

FIGS. 1A and 1B show a cross-sectional view of a low-pressure gas discharge lamp according to the invention,

FIG. 2 shows an emission spectrum of the low-pressure zirconium tetra-iodide gas discharge lamp,

FIG. 3 shows an emission spectrum of the low-pressure titanium tetra-iodide gas discharge lamp,

FIG. 4 shows an emission spectrum of the low-pressure hafnium tetra-iodide gas discharge lamp,

FIG. 5 shows an emission spectrum of the low-pressure zirconium tetra-chloride gas discharge lamp,

FIG. 6 shows an emission spectrum of the low-pressure hafnium tetra-chloride gas discharge lamp, and

FIG. 7 shows an emission spectrum of the low-pressure hafnium tetra-bromide gas discharge lamp.

The figures are purely diagrammatic and not drawn to scale. Particularly for clarity, some dimensions are exaggerated strongly. Similar components in the Figures are denoted by the same reference numerals as much as possible.

### DETAILED DESCRIPTION OF THE PREFERRED EMBODIMENTS

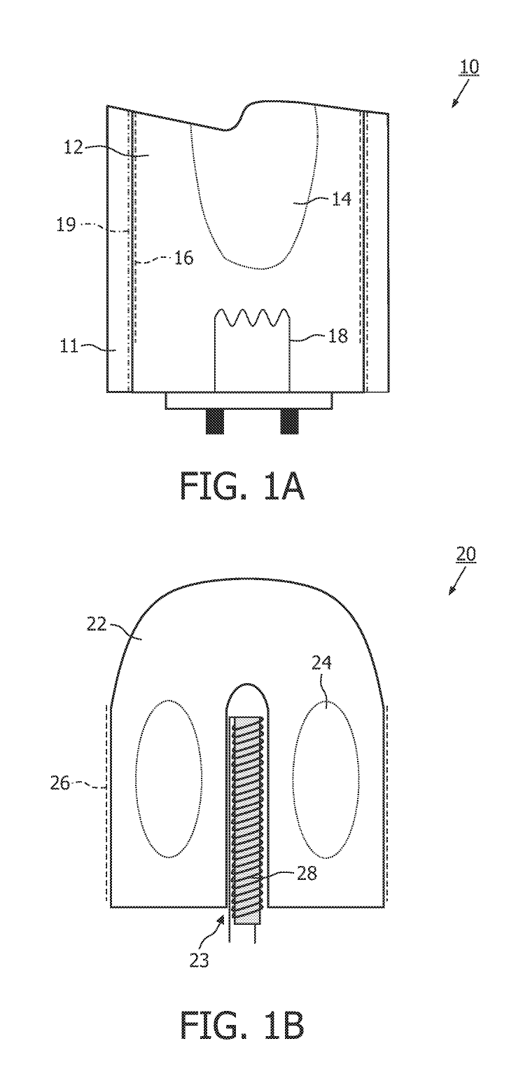

FIGS. 1A and 1B show a cross-sectional view of a low-pressure gas discharge lamp 10, 20 according to the invention. The low-pressure gas discharge lamp 10, 20 according to the invention comprises a light transmitting discharge vessel 12, 22 which encloses a discharge space 14, 24 in a gastight manner. The discharge space 14, 24 comprises a gas filling, for example, comprising a metal compound and a buffer gas. The low-pressure gas discharge lamp 10, 20 further comprises coupling elements 18, 28. The coupling elements, for example, couple energy into the discharge space 14, 24 via capacitive coupling, inductive coupling, microwave coupling, or via electrodes to obtain a gas discharge in the discharge space 14, 24.

In an embodiment shown in FIG. 1A the discharge elements 18 are a set of electrodes 18. In FIG. 1A only one electrode 18 of the set of electrodes 18 is shown. The electrodes 18 are electrical connections through the discharge vessel 12 of the low-pressure gas discharge lamp 10. By applying an electrical potential difference between the two electrodes 18 a discharge is initiated between the two electrodes 18. This discharge is generally located between the two electrodes 18 and is indicated in FIG. 1A as discharge space 14.

In general, light generation in the low-pressure gas discharge lamp 10 is based on the principle that charge carriers, particularly electrons but also ions, are accelerated by an electric field applied between the electrodes 18 of the low-pressure gas discharge lamp 10. Collisions of these accelerated electrons and ions with the gas atoms or molecules in the gas filling of the low-pressure gas discharge lamp 10 cause these gas atoms or molecules to be dissociated, excited or ionized. When the atoms or molecules of the gas filling return to the ground state, a more or less substantial part of the excitation energy is converted to radiation. The light emitted by the excited mercury atoms is mainly ultraviolet light at a wavelength of approximately 254 nanometer. This ultraviolet light is subsequently absorbed by a luminescent layer comprising a luminescent material which converts the absorbed ultraviolet light, for example, into visible light of a predetermined color.

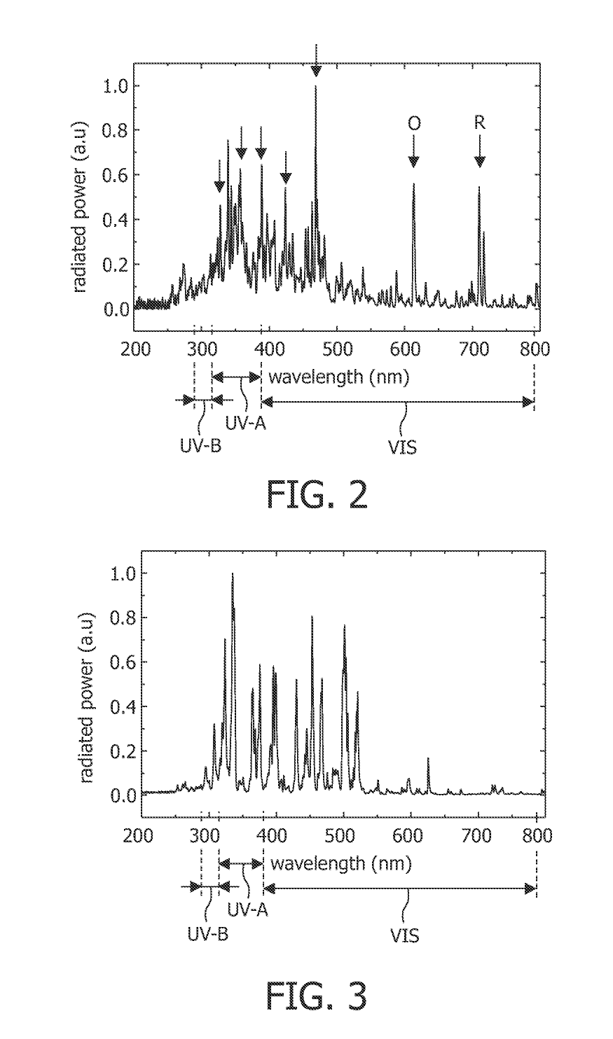

In the low-pressure gas discharge lamp 10 according to the invention, the discharge space 14 comprises a metal compound and a buffer gas. The metal compound in the low-pressure gas discharge lamp 10 according to the invention is selected from the group formed by compounds of titanium, zirconium, hafnium, and their mixtures. The buffer gas is generally constituted by an inert gas, for example, helium, neon, argon, krypton and/or xenon, preferably at a pressure between 0.1 and 100 mbar (typically at room temperature, not in operation). The metal compound added to the gas filling of the low-pressure gas discharge lamp 10 and its fragments in the discharge according to the invention are excited by the accelerated electrons and ions and subsequently emit light. The emission spectrum of the low-pressure gas discharge lamp 10 is determined by the type of metal compounds in the gas filling, together with, for example, the pressure and temperature inside the discharge vessel 12. The gas filling comprises different metal compounds, such as metal atoms and molecules, which all contribute to the emission spectrum of the low-pressure gas discharge lamp 10 according to the invention. For example, when adding titanium tetra-bromide to the gas filling of the low-pressure gas discharge lamp 10 according to the invention, the emission spectrum will be constituted by the titanium compounds and will comprise the characteristic titanium atom and ion emission lines together with the emission lines of the titanium molecules, such as titanium bromide, titanium di-bromide and/or titanium tetra-bromide. In FIGS. 2, 3 and 4 other examples of emission spectra are shown of metal compounds added to the gas filling of the low-pressure gas discharge lamp 10.

In the embodiment of the low-pressure gas discharge lamp 10 shown in FIG. 1A, a luminescent layer 16 is applied to the inside of the discharge vessel 12. The luminescent layer 16, for example, absorbs part of the near ultraviolet light emitted from the discharge space 14 and converts the absorbed ultraviolet light into visible light of a predetermined color. A few commonly used examples of luminescent materials from the vast range of possible luminescent materials are europium-activated barium magnesium aluminate, BaMgAl₁₀₁₇:Eu^{2 +} (also referred to as BAM) which emits substantially blue light, and europium-activated yttrium oxysulfide, Y₂O₂S:Eu (also referred to as YOS) which emits substantially red light. By choosing a specific luminescent material or mixture of luminescent materials, the color of the low-pressure gas discharge lamp 10 can be determined due to the mixing of the visible light emitted from the discharge space 14 with the light emitted by the luminescent layer 16.

The low-pressure gas discharge lamp 10, for example, also comprises a thermal insulation coating 19. Generally, an operating temperature of the low-pressure discharge lamps 10 according to the invention is higher compared to conventional low-pressure mercury vapor discharge lamps to ensure that enough metal vapor is in the gas filling. The additional thermal insulation coating 19, for example, is an infrared radiation-reflecting coating of Indium-doped Tin-Oxide (also known as ITO) or Fluor-doped Tin-Oxide (also known as FTO), reflecting the infrared radiation emitted from the discharge space 14 back towards the discharge space 14. The result of the added infrared radiation-reflecting coating is that the temperature inside the discharge vessel 12 increases.

The low-pressure gas discharge lamp 10, for example, also comprises an outer vessel 11 enclosing the discharge vessel 12. The additional outer vessel 11 provides additional thermal insulation, which further reduces the energy loss from loss of heat. Furthermore, a luminescent layer 16 may, for example, be applied at the inside of the outer vessel 11, preventing the luminescent material in the luminescent layer 16 from reacting with the gas filling inside the discharge vessel 12.

FIG. 1B shows an embodiment of the low-pressure gas discharge lamp 20 comprising an inductive coupler 28 for inductively maintaining the discharge in the low-pressure gas discharge lamp 20. Alternatively, the inductive coupler 28 may also be used for generating the discharge. The inductive coupler 28, also referred to as power coupler 28, generally comprises a coil wound over a ferrite core, for example Nickel-Zinc ferrite or Manganese-Zinc ferrite. The inductive coupler 28 is arranged in an indentation 23 in the discharge vessel 22 and generates a varying electromagnetic field inside the discharge vessel 22 at the discharge space 24. Electrons and ions in the gas filling of the discharge space 24 are accelerated by the electromagnetic field and collide with the metal compounds added to the gas filling. Due to the collision, the metal compounds are excited and subsequently emit light. The benefit of inductively generating and/or maintaining the discharge in the low-pressure gas discharge lamp 20 is that the electrodes 18, which generally limit the lifetime of low-pressure gas discharge lamps, can be omitted. Alternatively, the inductive coupler 28 may be arranged at the outside (not shown) of the discharge vessel 22, which results in a simplification of the manufacturing process for the discharge vessel 22. In the embodiment shown in FIG. 1B, the luminescent layer 26 is applied to the outside of the discharge vessel 22.

A selection of metal compounds which can be added to the low-pressure gas discharge lamp 10, 20 according to the invention can be found in table I. All listed metal compounds can be used both in general illumination applications and in UV-A and/or UV-B applications, which is indicated by means of “+”. However, some compounds are especially suitable for use in general illumination applications or for use in a UV-A and/or UV-B applications, which is indicated by means of “++”.

TABLE I
a selection of metal compounds according to the invention.

| | suitability of compound in | suitability of compound in |
|---|---|---|
| Compound | general illumination purposes | UVA/UVB application |
| TiCl₄ | ++ | ++ |
| TiBr₄ | ++ | ++ |
| TiI₄ | ++ | ++ |
| ZrCl₄ | ++ | + |
| ZrBr₄ | ++ | + |
| ZrI₄ | ++ | + |
| ZrOCl₂ | ++ | + |
| HfCl₄ | + | ++ |
| HfBr₄ | + | ++ |
| HfI₄ | + | ++ |
| HfOCl₂ | + | ++ |

FIG. 2 shows an emission spectrum of the low-pressure zirconium tetra-iodide gas discharge lamp 10, 20. The spectrum in FIG. 2 shows a clear combination of the emission lines of zirconium (some of the zirconium emission lines are indicated by means of an arrow in FIG. 2) and a continuous spectrum which ranges from approximately 250 nanometer to approximately 550 nanometer. The use of zirconium tetra-iodide is especially beneficial because the zirconium emission line at approximately 610 nanometers adds a color orange/red (indicated by means of O in FIG. 2) to the emission spectrum, and the zirconium emission line at approximately 710 nanometers adds a color red (indicated by means of R in FIG. 2) to the emission spectrum. Generally, luminescent material producing a red luminescence, for example, europium-activated yttrium oxysulfide, Y₂O₂S:Eu (also referred to as YOS) has a relatively low efficiency. The reason is that the Stokes shift for, for example, YOS is relatively large and thus the energy losses due to the Stokes shift are relatively large. The combination of the red R and orange O emission lines and the continuous spectrum, which mainly adds a blue color to the emission spectrum, results in substantially white light emitted from the low-pressure gas discharge lamp 10, 20 comprising a titanium compound, for example, titanium tetra-iodide, without the need for luminescent materials. In FIG. 2, the ranges of electromagnetic radiation defined as ultraviolet-B Nv-B (approximately between 280 nanometer and 320 nanometer), and ultraviolet-A UV-A (approximately between 320 nanometer and 400 nanometer), as well as the range of electromagnetic radiation defined as visible light VIS are indicated.

FIG. 3 shows an emission spectrum of the low-pressure titanium tetra-iodide gas discharge lamp 10, 20. As can be seen from FIG. 3, adding titanium tetra-iodide to the gas filling results in an emission of visible light especially in the blue range of the visible electromagnetic spectrum. To produce a low-pressure gas discharge lamp emitting substantially white light, either, for example, a luminescent layer 16, 26 should be added, or, for example, other metal compounds, such as titanium compounds, should be added to the gas filling to account for a red contribution to the emission spectrum. Titanium tetra-iodide provides a relatively strong emission in the UV-A and UV-B range and may beneficially be used in, for example, diagnostics applications such as medical imaging, therapeutic applications such as radiotherapy for the treatment of psoriasis, and cosmetic applications such as tanning applications.

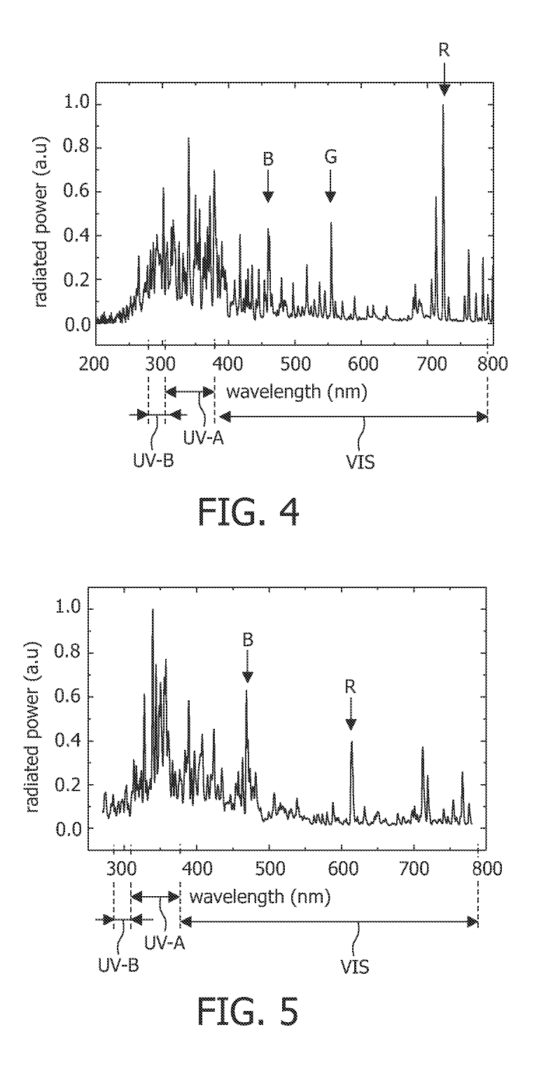

FIG. 4 shows an emission spectrum of the low-pressure hafnium tetra-iodide gas discharge lamp 10, 20. The continuous part of the emission spectrum of hafnium tetra-iodide is mainly in the ultraviolet-A UV-A and the ultraviolet-B UV-B range of the electromagnetic spectrum. The use of hafnium tetra-iodide is especially beneficial because the hafnium emission line at approximately 720 nanometer adds a deep red (indicated by means of R in FIG. 4) to the emission spectrum, enabling improved color rendering for the color red. However, as can be seen from the emission spectrum shown in FIG. 4, the contribution of the hafnium emission line at approximately 560 nanometers, adding a color green G to the emission spectrum, and the contribution of the hafnium emission line at approximately 460 nanometers, adding a color blue B to the emission spectrum, may not be sufficient to produce substantially white light. Adding, for example, a luminescent layer 16, 26, or, for example, mixing other metal compounds, such as hafnium compounds, into the gas filling may be required to obtain an emission of substantially white light from the low-pressure gas discharge lamp 10, 20 according to the invention.

FIG. 5 shows an emission spectrum of the low-pressure zirconium tetra-chloride gas discharge lamp 10, 20. Also for zirconium tetra-chloride, the continuous part of the emission spectrum is mainly in the ultraviolet-A UV-A and the ultraviolet-B UV-B range of the electromagnetic spectrum. The use of zirconium tetra-chloride in the low-pressure gas discharge lamp provides an emission line at approximately 470 nanometer, which adds a color blue (indicated by means of B in FIG. 5) to the emission spectrum, and an emission line at approximately 610 nanometer, which adds a color red (indicated by means of R in FIG. 5) to the emission spectrum. To obtain substantially white light emitted from the low-pressure zirconium tetra-chloride gas discharge lamp, for example, a green luminescent material may be used in a luminescent layer 16, 26, or, for example, other metal compounds such as hafnium compounds may be added to the gas filling.

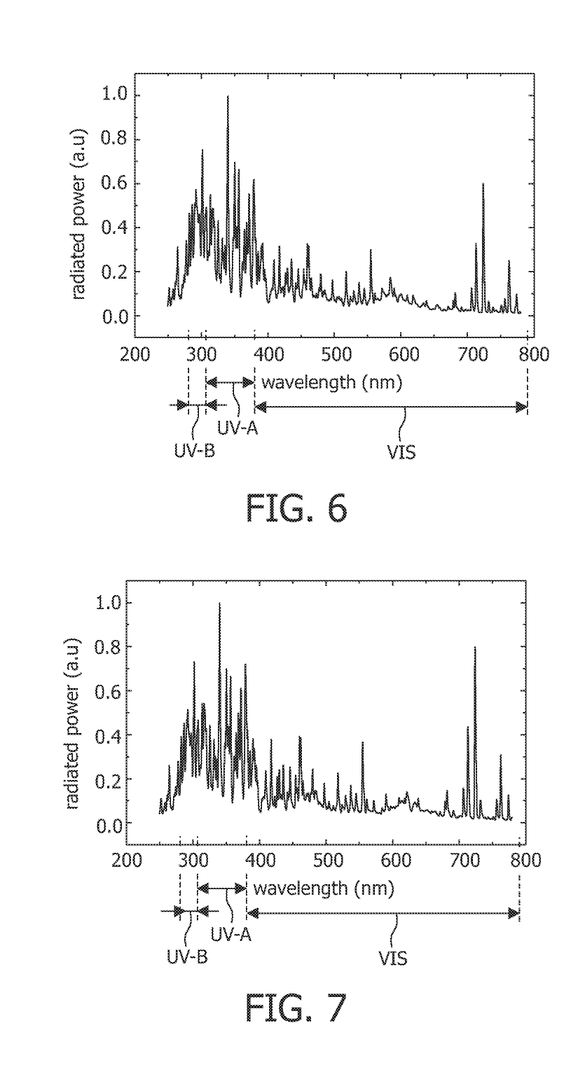

FIGS. 6 and 7 show the emission spectra of the low-pressure hafnium tetra-chloride gas discharge lamp 10, 20 and the low-pressure hafnium tetra-bromide gas discharge lamp 10, 20, respectively. Adding hafnium tetra-chloride or hafnium tetra-bromide to the gas filling results in similar emission spectra where the main emission, again, is in the ultraviolet-A UV-A and the ultraviolet-B UV-B range of the electromagnetic spectrum. As indicated in table I, the metal compounds hafnium tetra-chloride and hafnium tetra-bromide, added to the gas filling of the low-pressure gas discharge lamp, result in a low-pressure gas discharge lamp which is especially beneficial for UV-A and/or UV-B emission. Substantially white light for use in general illumination applications may be obtained by adding luminescent material in a luminescent layer 16, 26. Although luminescent materials are added to obtain substantially white light, the efficiency of the low-pressure hafnium tetra-chloride gas discharge lamp or the low-pressure hafnium tetra-bromide discharge lamp is typically increased with respect to low-pressure mercury vapor discharge lamps. The increased efficiency results from the substantially longer average wavelength of the ultraviolet part of the emitted light of the low-pressure hafnium tetra-chloride gas discharge lamp or the low-pressure hafnium tetra-bromide discharge lamp, compared to the main ultraviolet emission of the low-pressure mercury vapor discharge lamp. This increase of the average wavelength of the ultraviolet part of the emitted light in the low-pressure gas discharge lamps according to the invention reduces the energy loss due to the decreased Stokes shift compared to the known low-pressure mercury vapor discharge lamp.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements. In the device claim enumerating several means, several of these means may be embodied by one and the same item of hardware. The mere fact that certain measures are recited in mutually different dependent claims does not indicate that a combination of these measures cannot be used to advantage.

1. A low-pressure gas discharge lamp (10, 20) comprising:
a light-transmitting discharge vessel (12, 22) enclosing, in a gastight manner, a discharge space (14, 24) comprising a gas filling, the gas filling comprising a metal compound, and
the low-pressure gas discharge lamp (10, 20) further comprising discharge means (18, 28) for maintaining a discharge in the discharge space (14, 24),
the metal compound being selected from the group formed by compounds of titanium, zirconium, hafnium, and their mixtures.

2. Low-pressure gas discharge lamp (10, 20) as claimed in claim 1, wherein the gas filling of the discharge space (14, 24) further comprises a buffer gas.

3. Low-pressure gas discharge lamp (10, 20) as claimed in claim 1, wherein the metal compound comprises metal halides.

4. Low-pressure gas discharge lamp (10, 20) as claimed in claim 3, wherein the metal halides comprise metal tetra-halides and/or metal oxy-halides.

5. Low-pressure gas discharge lamp (10, 20) as claimed in claim 1, wherein, in operation, the density of the metal particles is between 5×1018 and 5×1024 particles/m3.

6. Low-pressure gas discharge lamp (10) as claimed in claim 1, wherein the low-pressure gas discharge lamp (10) comprises an outer vessel (11) enclosing the discharge vessel (12).

7. Low-pressure gas discharge lamp (10, 20) as claimed in claim 1, wherein the low-pressure gas discharge lamp (10, 20) is a substantially mercury-free low-pressure gas discharge lamp (10, 20).

8. Low-pressure gas discharge lamp (10, 20) as claimed in claim 1, wherein the discharge means (18, 28) maintain the discharge via inductive operation (28), or capacitive operation, microwave operation, or via electrodes (18).

9. Low-pressure gas discharge lamp (10, 20) as claimed in claim 1, wherein the discharge vessel (12, 22) comprises a luminescent layer (16, 26) comprising a luminescent material.

10. Low-pressure gas discharge lamp (10, 20) as claimed in claim 1, wherein the discharge vessel (12, 22) comprises a coating (19) for thermal insulation.

11. Use of the low-pressure gas discharge lamp (10, 20) as claimed in claim 1 for diagnostics, therapeutic, cosmetic and/or germicidal applications.
